(19) United States
(12) Patent Application Publication
(10) Pub. No.: US 2023/0348417 A1
(43) Pub. Date: Nov. 2, 2023

(54) PROCESS FOR PREPARING (S)-1-(1-ACRYLOYLPYRROLIDIN-3-YL)-3-((3,5-DIMETHOXYPHENYL) ETHYNYL)-5-(METHYLAMINO)-1H-PYRAZOLE-4-CARBOXAMIDE

(71) Applicant: BEIJING INNOCARE PHARMA TECH CO., LTD., Beijing (CN)

(72) Inventors: Yucheng PANG, Beijing (CN); Xiangyang CHEN, Beijing (CN)

(21) Appl. No.: 18/349,043

(22) Filed: Jul. 7, 2023

Related U.S. Application Data
(63) Continuation of application No. PCT/CN2022/071116, filed on Jan. 10, 2022.
(60) Provisional application No. 63/136,536, filed on Jan. 12, 2021.

(51) Int. Cl. C07D 401/04

(52) U.S. Cl. CPC C07D 401/04

(57) ABSTRACT

The present invention relates to processes for preparing (S)-1-(1-acryloylpyrrolidin-3-yl)-3-((3,5-dimethoxyphenyl)ethynyl)-5-(methylamino)-1H-pyrazole-4-carboxamide (Compound I) in a large scale of over 1 kg. The processes provide a good yield and a purity of at least 95% of Compound I which are safe and robust.

This application is a continuation of PCT/CN2022/071116, filed Jan. 10, 2022; which claims priority to U.S. Provisional Application No. 63/136,536, filed Jan. 12, 2021. The contents of the above-identified applications are incorporated herein by reference in their entirety.

### FIELD OF THE INVENTION

The present invention generally relates to a processes for preparing (S)-1-(1-acryloylpyrrolidin-3-yl)-3-((3,5-dimethoxyphenyl)ethynyl)-5-(methylamino)-1H-pyrazole-4-carboxamide, an FGFR inhibitor currently in clinical trials for the treatment of cancers, as well as novel intermediates used in the process.

### BACKGROUND OF THE INVENTION

(S)(1-acryloylpyrrolidin-3-yl)-3-((3,5-dimethoxyphenyl)ethynyl)-5-(methylamino)-1H-pyrazole-4-carboxamide (Compound I) is a potent inhibitor of Fibroblast Growth Factor Receptor (FGFR). Compound I is useful for treating cancers, inflammations, and other FGFR related diseases (WO2018/049781). WO2018/049781 (Example 21) discloses a synthetic route toward the preparation of about 1 g quantity of Compound I.

There is a need for efficient, scalable, and purity-controlled processes for preparing Compound I, particularly in a large scale of over 1 kg.

### DETAILED DESCRIPTION OF THE INVENTION

The present invention relates to a process for preparing (S)-1-(1-acryloylpyrrolidin-3-yl)-3-((3,5-dimethoxyphenyl)ethynyl)-5-(methylamino)-1H-pyrazole-4-carboxamide (Compound I) in high purity and good yield. The process is suitable for large-scale production (over 0.5 kg, preferably over 1 kg, over 2 kg, or over 5 kg). The process provides purity of Compound I ≥ 90%, or ≥ 95%, or ≥ 98%, or ≥ 99%.

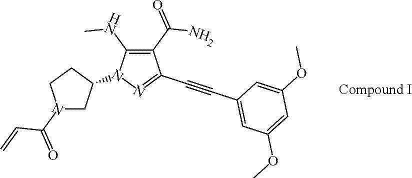 [SMILES: C=CC(=O)N1CC[C@H](n2nc(C#Cc3cc(OC)cc(OC)c3)c(C(N)=O)c2NC)C1]

In a first aspect, the invention provides a process for preparing Compound I. The process comprises the steps of:
(a) Sonogashira coupling between 4 and 7 in the presence of one or more suitable catalysts, one or more suitable bases, and one or more suitable solvents at 50~140° C. to afford 5,

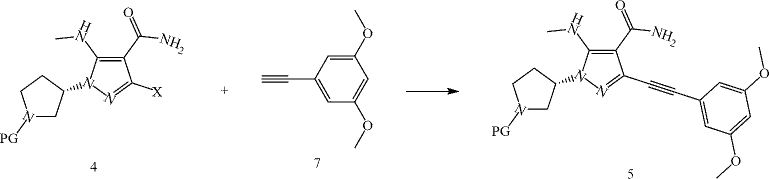

where PG is a protecting group of tert-butyloxycarbonyl (Boc), benzyloxycarbonyl (Cbz) or 2-(trimethylsilyl)ethoxymethyl (SEM), preferably Boc, and X is a leaving group, preferably Cl, Br or I.
(b) Deprotection of 5 with a suitable acid, for example HCl, in one or more suitable solvents to afford 6 or its salt, such as an HCl salt, to replace the PG

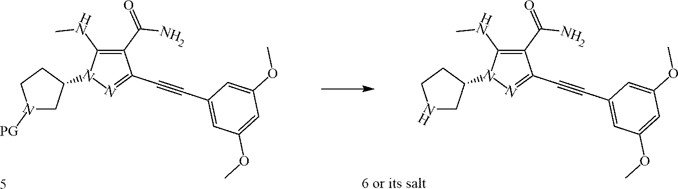

(c) Amidation of 6 or its salt with acryloyl chloride in the presence of one or more suitable bases in one or more suitable solvents to afford Compound I.

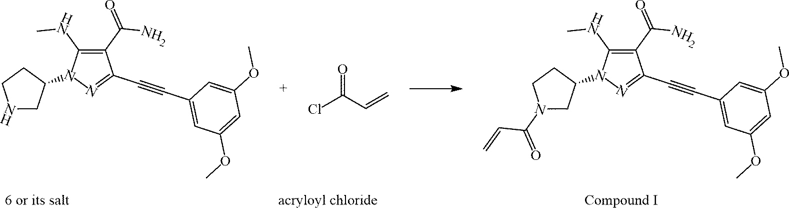

In step (a), Sonogashira coupling is performed to react a terminal alkyne with a heteroaryl halide in the presence of one to two catalysts and a base. For the conversion of 4 to 5, the starting materials 4 and 7, one or more suitable catalysts, and one or more suitable bases are mixed in one or more suitable solvents within a reactor under nitrogen with a low oxygen content (< 2%) to provide 5. The reaction temperature is 50-140° C., or 50-120° C., or 50-110° C., or 60-120° C., and preferably 65-85° C. The reaction time is typically 8 to 48 hours.

Suitable catalysts, as used in this application, can be chosen from palladium-containing catalysts, copper(I)-containing catalysts, or combinations of one or more palladium-containing catalysts and one or more copper(l)-containing catalysts. Palladium-containing catalysts include but are not limited to organopalladium compounds such as tris(dibenzylideneacetone)dipalladium(0) (Pd₂(dba)₃), tetrakis(triphenylphosphine)palladium(0) (Pd(PPh₃)₄), bis(triphenylphosphine)palladium(II) dichloride (Pd(PPh₃)₂Cl₂), [1,1′-bis(diphenylphosphino)ferrocene]dichloropalladium(II) (Pd(dppf)Cl₂), and inorganic palladium compounds, including Pd(OAc)₂ coordinated with various ligands, for example, Ph₃P, P(tBu)₃, PPh₂Cy, Cy₃P-HBF₄ and BINAP. Copper(I)--containing catalysts include but are not limited to Cu(I) salts such as Cu₂O, CuCl, CuBr, CuI and CuCN, etc. and preferably CuI.

Suitable bases, as used in step (a), include organic bases such as an amine base (for example, triethylamine (TEA), diisopropylethylamine (DIPEA), 1,8-diazabicyclo[5.4.0]undec-7-ene (DBU), N-methylmorpholine), pyridine, and substituted pyridine, and inorganic bases such as LiOH, NaOH, KOH, Na₂CO₃, NaHCO₃, K₂CO₃, Cs₂CO₃, KHCO₃, Li₃PO₄, Li₂HPO₄, Na₃PO₄, Na₂HPO₄, K₃PO₄, K₂HPO₄, LiF, NaF, KF, and CsF.

Suitable solvents, as used in step (a), can be chosen from organic solvents, water, or mixtures of one or more organic solvents and water. The organic solvents include but are not limited to tetrahydrofuran, 2-methyltetrahydrofuran, 1,4-dioxane, 1,2-dimethoxyethane, methanol, ethanol, isopropanol, n-butanol, benzene, toluene, xylene, DMF, DMA, NMP, DMSO, acetonitrile, EtOAc, iPrOAc, diethyl ether, methyl tert-butyl ether, dichloromethane, 1,2-dichloroethane, acetone, butan-2-one, etc.

In step (b), a suitable acid is added to remove the protecting group from 5 and provide 6, or its salt.

Suitable acids include strong acids such as HCl, HBr, HI, H₂SO₄, HClO₄, p-toluenesulfonic acid, trifluoroacetic acid. A preferred acid is HCl.

Suitable solvents, as used in step (b), can be chosen from but are not limited to tetrahydrofuran, 2-methyltetrahydrofuran, 1,4-dioxane, methanol, ethanol, toluene, EtOAc, iPrOAc, dichloromethane, 1,2-dichloroethane, acetone, etc.

In step (c), 6 or its salt is reacted with acryloyl chloride in one or more suitable solvents in the presence of a base at a temperature 0~30° C. for 1~24 hours to provide Compound I.

Suitable bases, as used in step (c), include organic bases such as an amine base (for example, triethylamine (TEA), diisopropylethylamine (DIPEA), and inorganic bases such as LiOH, NaOH, Na₂CO₃, NaHCO₃, K₂CO₃, Cs₂CO₃, KHCO₃, Li₃PO₄, Li₂HPO₄, Na₃PO₄, Na₂HPO₄, K₃PO₄, and K₂HPO₄.

Suitable solvents, as used in step (c), can be chosen from but are not limited to tetrahydrofuran, 2-methyltetrahydrofuran, 1,4-dioxane, EtOAc, iPrOAc, acetone, acetonitrile, water, etc.

In a second aspect, the invention provides an alternative process for preparing Compound I. The process is similar to the first process, except the sequential two steps of converting 6 to compound I. The alternative process comprises the steps of:
(a) Sonogashira coupling between 4 and 7 in the presence of one or more suitable catalysts, one or more suitable bases, and one or more suitable solvents at 50~140° C. to afford 5,

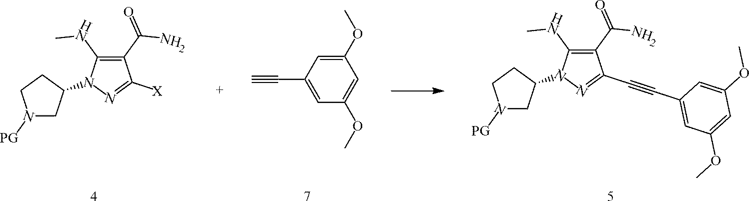

where PG is a protecting group of tert-butyloxycarbonyl (Boc), benzyloxycarbonyl (Cbz) or 2-(trimethylsilyl)ethoxymethyl (SEM), preferably Boc, and X is a leaving group, preferably Cl, Br or I.
(b) Deprotection of 5 with a suitable acid, for example HCl, in one or more solvents to afford 6 or its salt, such as an HCl salt.

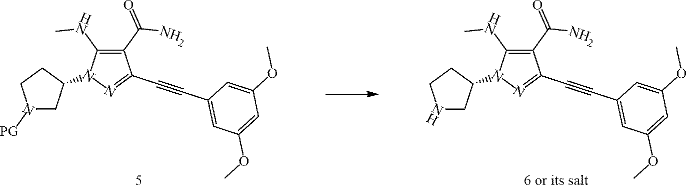

(c) Amidation of 6 or its salt with 3-chloropropanoyl chloride in the presence of one or more suitable bases in one or more suitable solvents to afford 8.

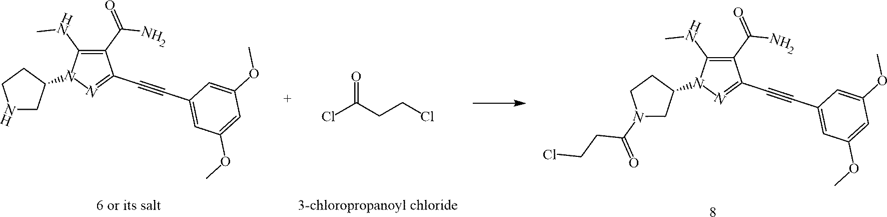

(d) 8 undergoes an elimination reaction with one or more suitable bases in one or more suitable solvents to afford Compound I.

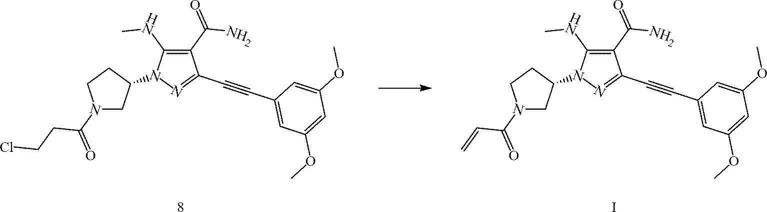

The details of steps (a) and (b) of the process of the second invention are the same as those of the first invention, while steps (c) and (d) are different.

In step (c), 6 or its salt is reacted with 3-chloropropanoyl chloride in one or more suitable solvents in the presence of a base at about -10-30° C. for 0.5-48 hours to provide 8.

Suitable bases, as used in step (c), include organic bases such as an amine base (for example, triethylamine (TEA), diisopropylethylamine (DIPEA)), and inorganic bases such as LiOH, NaOH, Na₂CO₃, NaHCO₃, K₂CO₃, Cs₂CO₃, KHCO₃, Li₃PO₄, Li₂HPO₄, Na₃PO₄, Na₂HPO₄, K₃PO₄, and K₂HPO₄.

Suitable solvents, as used in step (c), can be chosen from but are not limited to tetrahydrofuran, 2-methyltetrahydrofuran, 1,4-dioxane, EtOAc, iPrOAc, acetone, acetonitrile, water, etc.

In step (d), 8 undergoes an elimination reaction to remove H and Cl and form a double bond with one or more suitable bases in one or more suitable solvents at about -3-30° C. for 1-36 hours to afford Compound I.

Suitable bases, as used in step (d), include but are not limited to triethylamine (TEA), diisopropylethylamine, 1,8-diazabicyclo[5.4.0]undec-7-ene (DBU), LiOH, NaOH, KOH, Na₂CO₃, K₂CO₃, Cs₂CO₃, Li₃PO₄, Na₃PO₄ and K₃PO₄.

Suitable solvents, as used in step (d), can be chosen from but are not limited to tetrahydrofuran, 2-methyltetrahydrofuran, 1,4-dioxane, methanol, ethanol, EtOAc, iPrOAc, DMSO, DMF, DMA, NMP, acetone, acetonitrile, water, etc.

The present invention also provides a process for preparing the starting material 4 of the above two processes. The process comprises the steps of:
(a) Mitsunobu reaction of 1 and 9 in the presence of an azodicarboxylate reagent and an organophosphine compound in one or more suitable solvents to convert the alcohol 9 to 2,

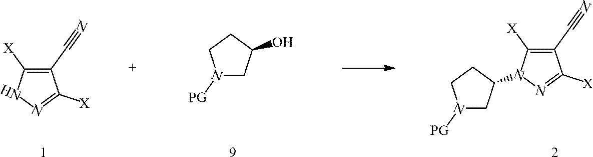

where PG is a protecting group of Boc, Cbz or SEM, preferably Boc, and X is a leaving group, preferably Cl, Br or I.
(b) 2 undergoes substitution reaction with MeNH₂ in one or more suitable solvents to afford 3.

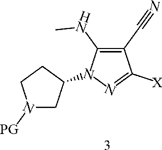

(c) Hydrolysis of 3 in the presence of H₂O₂ or urea hydrogen peroxide (H₂O₂·NH₂CONH₂) and one or more suitable bases in one or more solvents to afford 4.

In step (a) for the conversion of 1 to 2, the reaction between 1 and 9 is conducted in the presence of an azodicarboxylate reagent and an organophosphine compound. An azodicarboxylate reagent includes but is not limited to diethyl azodicarboxylate (DEAD), diisopropyl azodicarboxylate (DIAD) and di-tert-butyl azodicarboxylate (DBAD). An organophosphine compound includes but is not limited to triphenylphosphine, tricyclohexylphosphine and tributyl phosphine.

Suitable solvents, as used in step (a), can be chosen from but are not limited to tetrahydrofuran, 2-methyltetrahydrofuran, 1,4-dioxane, benzene, toluene, xylene, DMF, DMA, NMP, DMSO, acetonitrile, EtOAc, iPrOAc, etc.

Suitable solvents, as used in step (b), can be chosen from but are not limited to tetrahydrofuran, 2-methyltetrahydrofuran, 1,4-dioxane, MeOH, EtOH, acetonitrile, water, etc.

Suitable bases, as used in step (c), are preferred to be a strong base selected from but are not limited to LiOH, NaOH and KOH.

Suitable solvents, as used in step (c), can be chosen from but are not limited to 1,4-dioxane, MeOH, EtOH, acetonitrile, NMP, DMSO, water, etc.

The processes of the invention have several important advantages over prior synthesis of Compound I, fewer chemical steps, exclusive regio-selectivity, more efficiency, and higher overall yield. Additionally, the process consistently provides Compound I in high quality for use as a pharmaceutical API.

The present invention is further directed to the following compounds. The compounds are useful in the process for preparing Compound I.

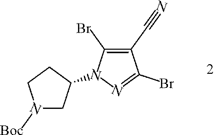 [SMILES: CC(C)(C)OC(=O)N1CC[C@H](n2nc(Br)c(C#N)c2Br)C1]

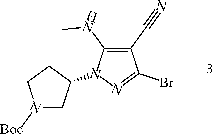 [SMILES: CNc1c(C#N)c(Br)nn1[C@H]1CCN(C(=O)OC(C)(C)C)C1]

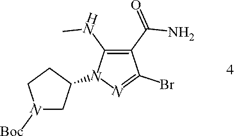 [SMILES: CNc1c(C(N)=O)c(Br)nn1[C@H]1CCN(C(=O)OC(C)(C)C)C1]

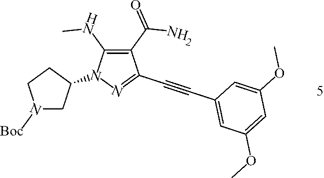 [SMILES: CNc1c(C(N)=O)c(C#Cc2cc(OC)cc(OC)c2)nn1[C@H]1CCN(C(=O)OC(C)(C)C)C1]

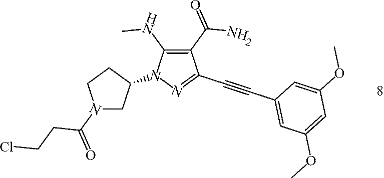 [SMILES: CNc1c(C(N)=O)c(C#Cc2cc(OC)cc(OC)c2)nn1[C@H]1CCN(C(=O)CCCl)C1]

The present invention is further illustrated by the following examples which are preferred embodiments of the invention. These examples are illustrative and are not meant to limit the possible techniques one skilled in the art may use to prepare compounds as disclosed herein.

### EXAMPLES

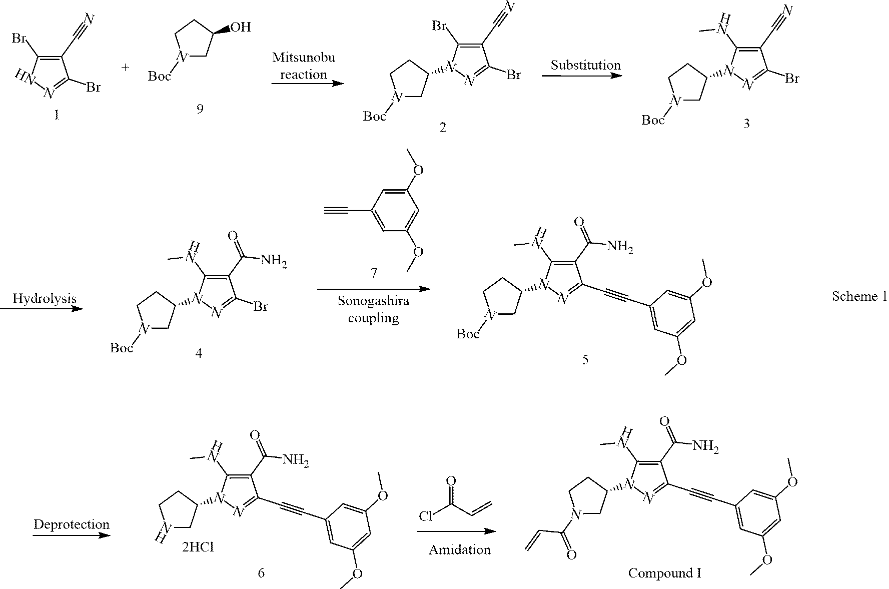

Scheme 1 summarizes a process of preparing Compound I, which is shown in Examples 1 to 6.

### Example 1. Preparation of Tert-Butyl (S)-3-(3,5-Dibromo-4-Cyano-1H-Pyrazol-1-yl)Pyrrolidine-1-Carboxylate (2)

To a 100-L reactor were added toluene (60 L, 13.3 L/kg), 1 (4.500 kg, 1.00 eq.), tert-butyl (R)-3-hydroxypyrrolidine-1-carboxylate (9, 3.358 kg, 1.00 eq.) and triphenylphosphine (5.410 kg, 1.15 eq.) at room temperature, and the resulting mixture was cooled to 5~10° C. while stirring. After addition of diisopropyl azodiformate (4.352 kg, 1.20 eq.) dropwise at 5~10° C. over 1.5 h, stirring was continued for 2 h at 0-10° C. (internal temperature) and then for 10 h at 25° C. The mixture was cooled to 5~10° C., filtered and washed with toluene (5 L, 1.11 L/kg). The filtrate was concentrated to dryness to which was added ethanol (15 L, 3.33 L/kg). The resulting mixture was then cooled to 20° C., stirred for 1 h and filtered. The solid was collected and dried under an infrared light to give 2 (6.000 kg, 80% yield).

### Example 2. Preparation of Tert-Butyl (S)-3-(3-Bromo-4-Cyano-5-(Methylamino)-1H-Pyrazol-1-yl)Pyrrolidine-1-Carboxylate (3)

To a 10-L autoclave were added THF (5.0 L), methylamine in methanol (25~30%, 6.5 L) and 2 (3.000 kg, 1.0 eq.). The mixture was heated to 80° C. for 12 h. After cooling to room temperature, the reaction mixture was transferred to a container. The above procedure was repeated once to get another batch of reaction mixture prepared from 2 (3.000 kg). The two batches were combined and concentrated to dryness. The residue was triturated with water (12 L) at room temperature for 1 h and then filtered. The filtered cake was washed with water (5 L) to give 3 (5.400 kg, wet).

### Example 3. Preparation of Tert-Butyl (S)-3-(3-Bromo-4-Carbamoyl-5-(Methylamino)-1H-Pyrazol-1-yl)Pyrrolidine-1-Carboxylate (4)

### (A) Scale of 3.2 Kg

To a 50-L reactor were added DMSO (25 L), NaOH (972.0 g, 5.00 eq.) and 3 (1.800 kg, 1.00 eq.). The mixture was cooled to 5~10° C. and then added with 30% H₂O₂ aqueous solution (6.385 kg, 13.40 eq.) dropwise in batches and the reaction was repeated twice. All three batches were combined, poured in ice water (the amount of ice water was 3 times of the volume of DMSO), and filtered. The filtered cake was washed with water (10 L) and then transferred to a 50-L reactor to which ethanol (12 L) was added. The resulting mixture was then heated to reflux and filtered while hot to remove insoluble solid. The filtrate was cooled to room temperature and the solid was filtered. The filtered cake was washed with ethanol (2 L) and dried in a fume hood in air to give 4 (3.228 kg, 57% yield, 99.6% purity).

### (B) Scale of 32 Kg

4 can also be prepared as the following procedure:

To a reactor were added DMSO (499 kg) and 3 (54 kg, 1.00 eq.). The mixture was cooled to 10~20° C. and then added with an aqueous solution of NaOH (prepared by mixing 5.54 kg of solid NaOH and 50.4 kg of water). The resulting mixture was stirred for 30 min at 10~20° C. then added with urea hydrogen peroxide (12.6 kg, 1.0 eq) and stirred for 4 hours. Addition of urea hydrogen peroxide and stirring sequence was repeated four times. The mixture was then added with water (1360.8 kg) when keeping the temperature at 10~20° C. and the resulting mixture was stirred for 2 hours. The mixture was centrifuged to collect the solid which was then washed with water (100.6 kg) and centrifuged again to collect the solid (44 kg). The solid was transferred to a reactor to which isopropanol (69.5 kg) and ethanol (39.68 kg) were added. The resulting mixture was then heated to 75~85° C. and stirred for 1~2 hours. The mixture was cooled to 20~30° C. with a rate of 8~12° C. per hour and stirred for 1 hour. The mixture was filtered and the filtered cake was dried in a vacuum oven at 40~50° C. for 12 h to give 4 (32 kg, 60% yield, 99.3% purity).

### Example 4. Preparation of Tert-Butyl (S)-3-(4-Carbamoyl-3-((3,5-Dimethoxyphenyl)Ethynyl)-5-(Methylamino)-1H-Pyrazol-1-yl)Pyrrolidine-1-Carboxylate (5)

A reactor was charged with 2-methyltetrahydrofuran (15.200 kg, 10 L/kg), bubbled with N₂ for 1 h, and then added with 4 (1.765 kg, 1.00 eq.), 7 (0.895 kg, 1.21 eq.), NaHCO₃ (0.570 kg, 1.49 eq.), CuI (4.50 g, 0.005 eq.) and deionized water (17.700 kg, 10 L/kg) sequentially under N₂. The reactor was evacuated under vacuum and flushed with N₂ three times. Pd(PPh₃)₄ (0.270 kg, 0.05 eq.) was then added and the resulting mixture was heated to reflux for 16~20 h. The reaction mixture was cooled to 20-30° C., and the organic phase was washed with 7% sodium bicarbonate solution (17.805 kg, 10 L/kg) and 10% sodium chloride solution (18.000 kg, 10 L/kg) sequentially, and the combined aqueous phase was extracted with 2-methyltetrahydrofuran (5.305 kg, 3.5 L/kg). To the combined organic phase were added 1,3,5-triazine-2,4,6-(1H,3H,5H)-trithione trisodium salt (TMT-Na3) (0.725 kg, 0.64 eq.) and activated carbon (0.735 kg, 0.42 kg/kg), and the resulting mixture was heated to 40~50° C. for 12~18 h while stirring. After cooling to 20-30° C., the mixture was filtered through a pad of Celite (0.895 kg, 0.5 1 kg/kg). The filtered cake was washed with 2-methyltetrahydrofuran (1.750 kg, 1 L/kg), and the filtrates were combined and washed with 7% NaHCO₃ solution (17.750 kg, 10 L/kg), followed by 10% NaCl solution (18.455 kg, 10 L/kg). The aqueous solutions were combined and extracted with 2-methyltetrahedronfuran (5.300 kg, 3.5 L/kg). The organic phases were combined and concentrated. The residue was co-evaporated with EtOAc (9 kg, 5.6 L/kg) three times and then mixed with EtOAc (12.9 kg, 8.1 L/kg). The resulting solution was used directly in Example 5 without further purification.

### Example 5. Preparation of (S)-3-((3,5-Dimethoxyphenyl)Ethynyl)-5-(Methylamino)-1-(Pyrrolidin-3-yl)-1H-Pyrazole-4-Carboxamide Hydrochloride (6)

The solution from Example 5 was cooled to 15~25° C. to which was added a solution of HCl in EtOH (3.990 kg, 2.26 kg/kg) dropwise. The resulting was stirred for 1~5 h, filtered, and washed with EtOAc (0.360 kg, 0.2 L/kg). The filtered cake was then triturated with EtOAc (3.360 kg, 2.6 L/kg) and acetone (3.605 kg, 2.6 L/kg) sequentially. The wet solid was collected, and vacuum dried at 40~50° C. for 18-24 h to give 6 (1.765 kg, 88% yield over two steps, 99.3% purity).

### Example 6. Preparation of (S)-1-(1-Acryloylpyrrolidin-3-yl)-3-((3,5-Dimethoxyphenyl)Ethynyl)-5-(Methylamino)-1H-Pyrazole-4-Carboxamide (Compound I)

To a reactor were added 2-methyltetrahydrofuran (14.005 kg, 9.4 L/kg), 6 (1.760 kg, 1.0 eq.), 2,6-di-tert-butyl-4-methylphenol (DHT) (6.50 g, 0.006 eq.) and water (17.800 kg, 10 L/kg) under N₂ at 15~25° C., followed by addition of NaHCO₃ (1.460 kg, 4.0 eq.) portion-wisely. The resulting mixture was then cooled to 0~10° C. to which was added a solution of acryloyl chloride (394 g, 1.0 eq.) in 2-methyltetrahydrofuran (1.600 kg) via a dropping funnel. After the completion of addition, the resulting mixture was stirred at 0~10° C. for 1~2 h and then warmed to 10~20° C. for 15-45 min. The aqueous phase was separated, and the organic phase was washed with 7% NaHCO₃ aqueous solution (17.790 kg, 10 L/kg) and 10% NaCl aqueous solution (17.610 kg, 10 L/kg) sequentially. The aqueous phases were combined and extracted with 2-methyltetrahydrofuran (5.305 kg, 3.5 L/kg). The organic phases were combined and filtered through a pad of activated carbon (350.20 g, 0.20 kg/kg). The filtrate was concentrated to 6-8 kg and the residue was co-evaporated with EtOAc (8.8 kg, 5.5 L/kg) three times to 6-8 kg. The resulting EtOAc solution was cooled to 10-15° C. to which n-Heptane (7.910 kg, 6.6 L/kg) was added dropwise over 1~3 h and stirred for 1~3 h. The resulting mixture was cooled to 0-5° C., stirred for 1~5 h and filtered. The solid was triturated with a mixture of EtOAc (1.450 kg, 0.9 kg/kg) and n-heptane (1.450 kg, 0.9 kg/kg), and filtered. The wet solid was collected, and vacuum dried at 40-50° C. for 12~20 h to afford Compound I (1.345 kg, 80%yield, 98.8% purity). Compound I (1.340 kg, 1 eq.) was further purified by recrystallization from acetone (6.030 kg, 5.7 L/kg), water (4.020 kg, 3.0 Ukg) containing DHT (4.68 g, 0.006 eq.) under N₂ to afford purer Compound I (1.175 kg, 88% yield, 99.5% purity).

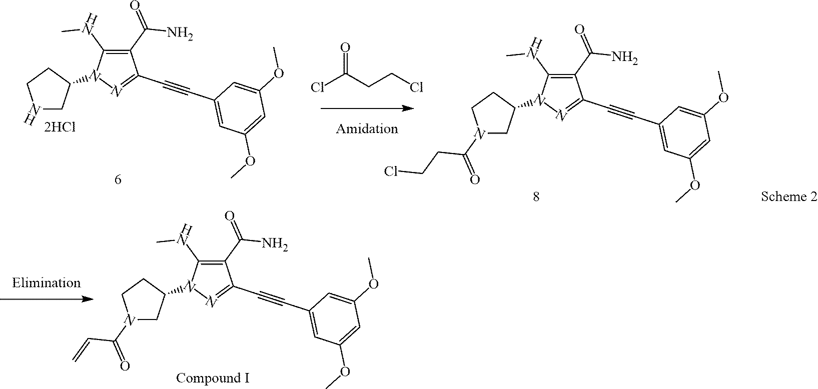

Scheme 2 summarizes a sequential two-step process for conversion of 6-HCl salt to Compound I, which is shown in Examples 7 and 8.

### Example 7. Preparation of (S)-1-(1-(3-Chloropropanoyl)Pyrrolidin-3-yl)-3-((3,5-Dimethoxyphenyl)Ethynyl)-5-(Methylamino)-1H-Pyrazole-4-Carboxamide (8)

To a 200-L reactor were added tetrahydrofuran (50 kg, 10 L/kg) and an aqueous solution of sodium bicarbonate (prepared by dissolving solid sodium bicarbonate (4.26 kg, 4.0 eq.) in deionized water (56 kg, 10 L/kg)), followed by 6 (5.60 kg, 1.0 eq.) through a spray solid addition funnel. After cooling to -5~5° C., a solution of 3-chloropropionyl chloride (1.778 kg, 1.0 eq.) in tetrahydrofuran (17.45 kg, 4 L/kg) was slowly dropped into the reactor at a rate of 8~12 kg/h and the apparatus was rinsed with tetrahydrofuran (4.98 kg, 1 L/kg) upon addition. The mixture was stirred at -5~5° C. for 0.5 h, warmed up to 20-30° C., and separated. The aqueous phase was extracted with 2-methyltetrahydrofuran (11.03 kg, 2.3 L/kg). The combined organic phase was washed with saturated sodium chloride solution (37.80 kg, 5.0 L/kg) and concentrated under reduced pressure at ≤ 40° C. to 11.20-22.40 L (2-4 L/kg). The residue was co-evaporated with ethyl acetate (28.00 kg, 5.5 L/kg) three times, concentrated to 20-30 L (3.6~5.4 L/kg) and added with ethyl acetate (25.20 kg, 5.0 L/kg). The resulting mixture was stirred at 15~25° C. for 16 h and filtered. The filtered cake was rinsed with ethyl acetate (5.55 kg, 1.1 L/kg) and dried under vacuum at 40-50° C. to give 8 (3.96 kg, 60% yield, 99.6% purity).

### Example 8. Preparation of (S)-1-(1-Acryloylpyrrolidin-3-yl)-3-((3,5-Dimethoxyphenyl)ethynyl)-5-(Methylamino)-1H-Pyrazole-4-Carboxamide (Compound I)

To an 80-L reactor were added acetonitrile (34.10 kg, 11 L/kg) and deionized water (7.90 kg, 2.0 L/kg). The mixture was adjusted to 15~25° C. and added with 8 (3.90 kg, 1.0 eq.) through a spray solid addition funnel. The funnel was then rinsed with acetonitrile (3.10 kg, 1 L/kg) into the reactor. The resulting solution was then transferred to a 500-L reactor with additional acetonitrile (3.10 kg, 1 L/kg) used for rinsing the 80-L reactor. The temperature of the reactor was adjusted to 15~25° C. and a solution of sodium hydroxide (prepared by dissolving NaOH (0.68 kg, 2 eq.) in water (7.80 kg, 2.0 L/kg) was added at a rate of 9~18 kg/h. The reaction mixture was stirred at 15~25° C. for 4.0 h. After cooling to 10~20° C., to the reactor was added slowly a diluted hydrochloric acid aqueous solution (prepared by mixing 0.85 kg of concentrated hydrochloric acid and 8.00 kg of deionized water) to adjust pH to 7~8. The temperature was adjusted to 20~30° C. and the mixture was separated. The organic phase was concentrated to 18~27 L at ≤ 40° C. and added with acetone (15.36 kg, 5.0 L/kg). The resulting mixture was heated to 35~40° C. till all the solid was dissolved. Deionized water (77.96 kg, 20 L/kg) was slowly added at a rate of 20-40 kg/h and the temperature was maintained at 33-42° C. After addition of water, the mixture was stirred at 33-42° C. for 1 hour, slowly cooled to 0~10° C. at a rate of 5~10° C./h, stirred for 3~5 h and filtered. The filtered cake was dried under vacuum at 40-50° C. to give Compound I (3.30 kg, 91.9% yield, 99.8% purity).

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method for preparing (S)-1-(1-acryloylpyrrolidin-3-yl)-3-((3,5-dimethoxyphenyl)ethynyl)-5-(methylamino)-1H-pyrazole-4-carboxamide (Compound I) comprising the steps of:
(a) reacting 4 and 7 in the presence of one or more catalysts and a first base in one or more suitable solvent at 50-140° C. to afford 5, 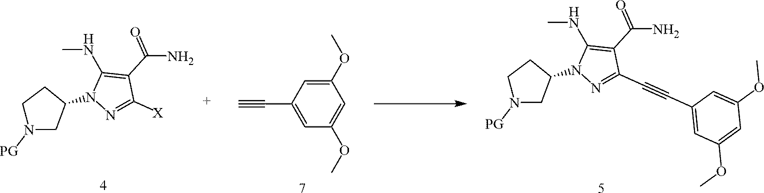
where PG is a protecting group of tert-butyloxycarbonyl (Boc), benzyloxycarbonyl(Cbz), or 2-(trimethylsilyl)ethoxymethyl (SEM), and X is a leaving group of Cl, Br or I;
(b) removing the protecting group in 5 with an acid to afford 6 or its salt; 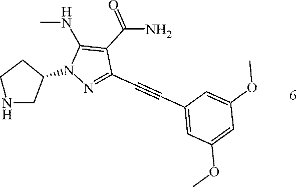 [SMILES: CNc1c(C(N)=O)c(C#Cc2cc(OC)cc(OC)c2)nn1[C@H]1CCNC1]
(c) reacting 6 or its salt with acryloyl chloride in the presence of a second base to afford Compound I; 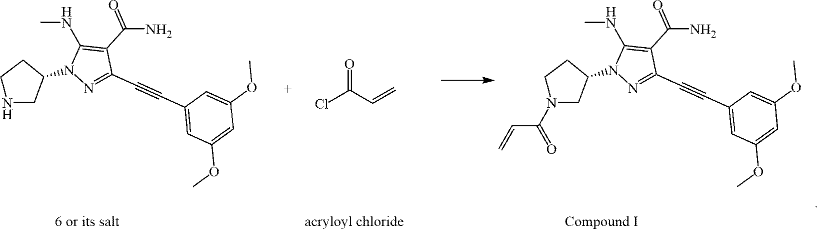
.

2. A method for preparing (S)-1-(1-acryloylpyrrolidin-3-yl)-3-((3,5-dimethoxyphenyl)ethynyl)-5-(methylamino)-1H-pyrazole-4-carboxamide (Compound I) comprising the steps of:
(a) reacting 4 and 7 in the presence of one or more catalysts and a first base in one or more suitable solvent at 50-140° C. to afford 5, 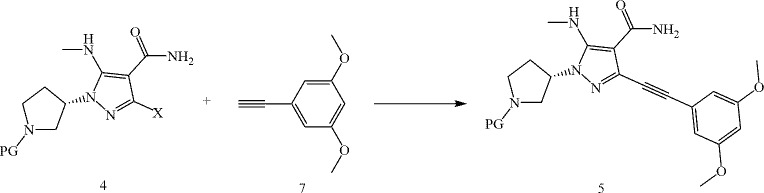
where PG is a protecting group of tert-butyloxycarbonyl (Boc), benzyloxycarbonyl (Cbz), or 2-(trimethylsilyl)ethoxymethyl (SEM), and X is a leaving group of Cl, Br or I;
(b) removing the protecting group in 5 with an acid to afford 6 or its salt; 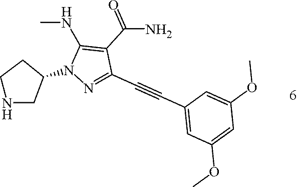 [SMILES: CNc1c(C(N)=O)c(C#Cc2cc(OC)cc(OC)c2)nn1[C@H]1CCNC1]
(c) reacting 6 or its salt with 3-chloropropanoyl chloride in the presence of a second base to afford to afford 8; 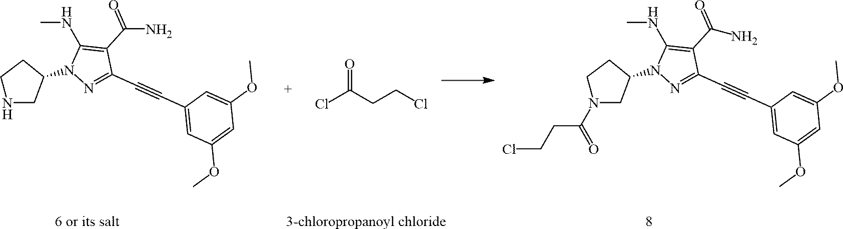
and
(d) reacting 8 with a second base to afford Compound I 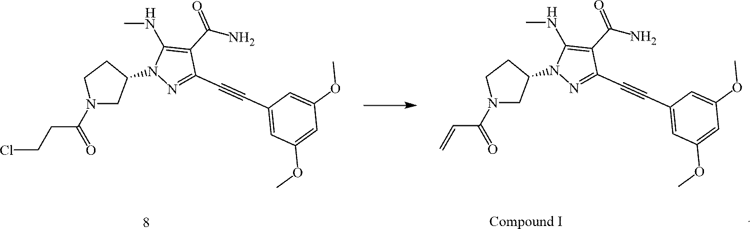
.

3. The method according to claim 1, wherein the one or more catalysts in step (a) are selected from the group consisting of palladium-containing catalysts, copper(I)-containing catalysts, and any combination thereof.

4. The method according to claim 3, wherein the palladium-containing catalyst is tris(dibenzylideneacetone)dipalladium(0) (Pd₂(dba)₃), tetrakis(triphenylphosphine)palladium(0) (Pd(PPh₃)₄), bis(triphenylphosphine)palladium(II) dichloride (Pd(PPh₃)₂Cl₂), or [1,1′-bis(diphenylphosphino)ferrocene]dichloropalladium(II) (Pd(dppf)Cl₂).

5. The method according to claim 3, wherein the palladium-containing catalyst is an inorganic palladium compound.

6. The method according to claim 3, wherein the copper(I)-containing catalyst is Cu₂O, CuCl, CuBr, CuI, or CuCN.

7. The method according to claim 1, wherein the acid in step (b) is a strong acid.

8. The method according to claim 7, wherein the acid is HCl, HBr, HI, H₂SO₄, HClO₄, p-toluenesulfonic acid, or trifluoroacetic acid.

9. The method according to claim 2, wherein the one or more catalysts in step (a) are selected from the group consisting of palladium-containing catalysts, copper(I)-containing catalysts, and any combination thereof.

10. The method according to claim 9, wherein the palladium-containing catalyst is tris(dibenzylideneacetone)dipalladium(0) (Pd₂(dba)₃), tetrakis(triphenylphosphine)palladium(0) (Pd(PPh₃)₄), bis(triphenylphosphine)palladium(II) dichloride (Pd(PPh₃)₂Cl₂), or [1,1′-bis(diphenylphosphino)ferrocene]dichloropalladium(II) (Pd(dppf)Cl₂).

11. The method according to claim 9, wherein the palladium-containing catalyst is an inorganic palladium compound.

12. The method according to claim 9, wherein the copper(I)-containing catalyst is Cu₂O, CuCl, CuBr, CuI, or CuCN.

13. The method according to claim 9, wherein the acid in step (b) is a strong acid.

14. The method according to claim 13, wherein the acid is HCl, HBr, HI, H₂SO₄, HClO₄, p-toluenesulfonic acid, or trifluoroacetic acid.

15. A method for preparing Compound 4, comprising the steps of:
(a) reacting 1 and 9 in the presence of an azodicarboxylate reagent and an organophosphine compound to afford 2, 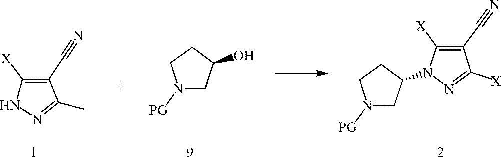
where PG is a protecting group of Boc, Cbz or SEM, and X is a leaving group of Cl, Br or I.
(b) reacting 2 with CH₃NH₂ to afford 3. 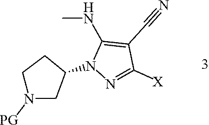
(c) hydrolyzing 3 in a solution comprising H₂O₂ or urea hydrogen peroxide and a base to afford 4. 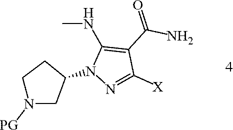
.

16. The method according to claim 15, wherein the azodicarboxylate reagent is diethyl azodicarboxylate (DEAD), diisopropyl azodicarboxylate (DIAD), or di-tert-butyl azodicarboxylate (DBAD).

17. The method according to claim 15, wherein the organophosphine compound is triphenylphosphine, tricyclohexylphosphine, or tributyl phosphine.

18. A compound selected from the group consisting of: 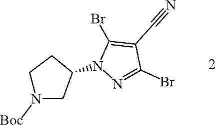 [SMILES: CC(C)(C)OC(=O)N1CC[C@H](n2nc(Br)c(C#N)c2Br)C1] 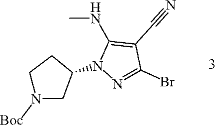 [SMILES: CNc1c(C#N)c(Br)nn1[C@H]1CCN(C(=O)OC(C)(C)C)C1] 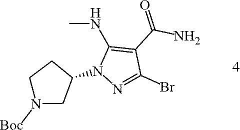 [SMILES: CNc1c(C(N)=O)c(Br)nn1[C@H]1CCN(C(=O)OC(C)(C)C)C1] 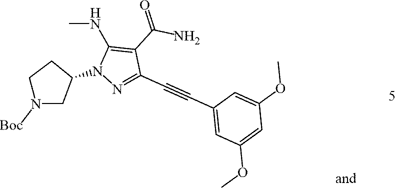 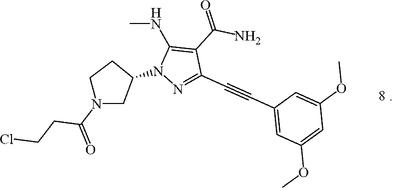 [SMILES: CNc1c(C(N)=O)c(C#Cc2cc(OC)cc(OC)c2)nn1[C@H]1CCN(C(=O)CCCl)C1]
